(19) United States
(12) Patent Application Publication
(10) Pub. No.: US 2017/0136100 A1
(43) Pub. Date: May 18, 2017

(54) Thioredoxin or Thioredoxin Derivatives or Peptides for the Treatment of High Blood Pressure

(71) Applicant: TEXAS TECH UNIVERSITY SYSTEM, Lubbock, TX (US)

(72) Inventor: Kumuda Das, Tyler, TX (US)

(21) Appl. No.: 15/323,545

(22) PCT Filed: Jun. 23, 2015
(86) PCT No.: PCT/US2015/037131

§ 371 Date: Jan. 3, 2017

Related U.S. Application Data
(60) Provisional application No. 62/020,146, filed on Jul. 2, 2014.

(51) Int. Cl. A61K 38/44

(52) U.S. Cl. CPC A61K 38/44; C12Y 108/01009

(57) ABSTRACT

The present invention provides methods and compositions for the treatment of cardiovascular disorders comprising: a therapeutically effective amount of thioredoxin-1 or a thioredoxin-1 derivative in a pharmaceutical carrier to ameliorate one or more symptom of a cardiovascular disorder.

### FIELD OF INVENTION

The present invention relates in general to the field of cardiovascular disorders, and more particularly, to hypertension and thioredoxin and thioredoxin derivatives or peptides for treatment of high blood pressure.

### BACKGROUND ART

Without limiting the scope of the invention, its background is described in connection with hypertension treatments. Hypertension is a common lifelong disease where the arterial pressure is elevated to an undesired level. Globally, hypertension is very common in both developed and developing countries. This chronic disease affects an increasing number of Americans each year and according to the American Heart Association, high blood pressure was listed as a primary or contributing cause of death in roughly 348,000 of the more than 2.4 million U.S. deaths in 2009. Prevalence of the disease varies largely by age, sex, and ethnicity. Hypertension is a major contributor to many cardiovascular disorders, including ventricular dysfunction, coronary artery disease, and heart failure. Aging is an independent factor for the onset of hypertension; in fact, people who are non-hypertensive at 55 years-of-age have a 90% lifetime risk of eventually developing the disease. Although a number of biochemical processes (e.g., oxidative stress, reduced nitric oxide, high renin-angiotensin activity, and endothelial dysfunction) have been identified as contributors to hypertension, the fundamental mechanisms involved in blood pressure control in aging are not completely understood.

### SUMMARY OF THE INVENTION

The present invention provides a composition for the treatment of a hypertensive disorder comprising: a therapeutically effective amount of thioredoxin-1 or a thioredoxin-1 derivative in a pharmaceutical carrier to ameliorate one or more symptoms associated with the hypertensive disorder. The pharmaceutical carrier may be adapted for intramuscular, intraperitoneal, intravenous, or subcutaneous delivery.

The present invention also provides a composition for the treatment of a cardiovascular disorder comprising: a therapeutically effective amount of thioredoxin-1 or a thioredoxin-1 derivative in a pharmaceutical carrier to ameliorate one or more symptoms of a cardiovascular disorder. The cardiovascular disorders may be a hypertensive disorder.

The present invention provides a pharmaceutical composition for the treatment of a cardiovascular disorder comprising: a therapeutically effective amount of a thioredoxin-1, a thioredoxin-1 derivative or combination thereof in a pharmaceutical carrier to ameliorate one or more symptoms of a cardiovascular disorder.

The present invention also provides a method for treating a patient having a cardiovascular disorder by administering to the patient therapeutically effective amounts of thioredoxin or a thioredoxin-1 derivative in a pharmaceutical carrier to ameliorate one or more symptoms of a cardiovascular disorder. The pharmaceutical carrier may be adapted for intramuscular, intraperitoneal, intravenous, or subcutaneous delivery and delivered every 0.5, 1, 2, 3, 4, 5, 6, or more months.

The present invention provides a method for treating a patient having a cardiovascular disorder by administering to the patient therapeutically effective amounts of thioredoxin system upregulator or activator and/or of an upregulator or activator of thioredoxin system and of an agent causing depletion of nitric oxide in the body.

The present invention provides a method for preserving aging-induced endothelial function in a patient comprising the steps of: administering to the patient a therapeutically effective amount of thioredoxin, a thioredoxin-1 derivative, a thioredoxin system upregulator, or a thioredoxin system activator in a pharmaceutical carrier to preserve aging-induced endothelial function.

The present invention provides a method for activation of eNOS protein function in a patient comprising the steps of: administering to the patient therapeutically effective amounts of thioredoxin, a thioredoxin-1 derivative, a thioredoxin system upregulator, or a thioredoxin system activator in a pharmaceutical carrier to activate eNOS protein function.

### DESCRIPTION OF THE DRAWINGS

For a more complete understanding of the features and advantages of the present invention, reference is now made to the detailed description of the invention along with the accompanying figures and in which:

FIG. 1 shows Trx-Tg mice are phenotypes that show decreased blood pressure. The basal blood pressure measurements show that Trx-Tg mice have lower blood pressure than the wildtype or mice expressing mutant thioredoxin.

FIG. 2 shows thioredoxin lowers angiotensin II mediated hypertension in mice.

FIG. 3 shows the entire spectrum of 14 day blood pressure measurement that shows that in each time point the blood pressure of Trx-Tg mice is significantly lower.

FIG. 4 shows the mesenteric artery isolated from the Trx-Tg mice show significantly higher relaxation response compared to wildtype or mutant mice.

FIG. 5 is an image showing the redox state of vessels in WT, Trx-Tg and dnTrx-Tg in both young and old mice.

FIGS. 6A-6F show the SMA outward remodeling and depolarization- and agonist-induced contraction is maintained in Trx-Tg mice.

FIGS. 7A-7D show aging-induced endothelial dysfunction is suppressed in Trx-Tg mice compared to WT and dnTrx-Tg mice.

FIGS. 8A-8D show preserved NO-mediated relaxing responses and EDH-mediated responses in young and aged Trx-Tg mice, WT and dnTrx-Tg mice.

FIGS. 9A 9B show Trx enhances ACh-mediated NO release in SMA from young and aged mice.

FIGS. 10A-10F show aging-induced endothelial dysfunction is restored by exogenous DTT and catalytically active h-Trx-1 in SMA from WT mice.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the abbreviation “SMA” denotes a superior mesenteric artery.

As used herein the abbreviation “PHE” denotes phenylephrine.

Accumulating evidence suggests that endothelial nitric oxide synthase (eNOS) that produces nitric oxide (NO), a critical endothelial derived relaxing factor which becomes dysfunctional during aging. A dysfunctional eNOS produces superoxide anion (O2._) instead of NO. Consistent with the fact that aging is a phenomenon with accumulated oxidative products over the life-span of an organism, eNOS remains in a more oxidized state with decreased potential to produce NO. Therefore, lack of adequate NO in aging causes decreased vasorelaxation that is considered as a major mechanism of age-related hypertension and arterial stiffness. Consequently, increased endothelial dysfunction is experienced in aged individuals that affect overall blood pressure control.

Elderly populations are at a very high risk of cardiovascular disorders due to an added factor of age-related hypertension. Because aging is manifested with cumulative oxidative products. Cytosolic thioredoxin (Trx) is a small (12 kDa) antioxidant protein that protects against oxidative stress. As a reducing agent, it regenerates proteins and enzymes inactivated by oxidation. Considering that oxidative stress is a major cause of hypertension, endothelial dysfunction, and loss of vascular tone, Trx provides a therapeutic composition that can protect aging individuals against hypertension. The present invention provides a transgenic mouse line that is deficient in functional thioredoxin (dnTrx-Tg), and a complementary line that over expresses the human protein (Trx-Tg), by mutating its redox-active Cys32-Cys35 Trx residues to serine; these mice (dnTrx-Tg) maintain only low levels of active Trx. The younger mice of all genotypes had normal blood pressure and endothelial function with Trx-Tg mice. However, older (>2 years) dnTrx-Tg and wildtype mice showed markedly decreased arterial relaxation, while aged-Trx-Tg mice continued to function normally. Functional NO release in en face SMA, as measured with the green fluorescent NO probe DAF-FM, was uncompromised in Trx-Tg mice, but severely reduced in WT or dnTrx-Tg mice. In WT and dnTrx-Tg mice, aging was accompanied by a reduced dimer:monomer ratio and increased eNOS monomerization, which were maintained in Trx-Tg mice. Thus, in terms of hypertension, the properties of aged Trx-Tg mice resemble younger animals. The chemical reductant dithiotreitol (100 mM) was able to restore the aging-induced endothelial dysfunction in WT, partially in aged dnTrx-Tg, but was ineffective in SMA from aged Trx-Tg, which expressed reduced Trx. These results provide compelling evidence that preservation of vessel redox status in aged mice protects against endothelial dysfunction and maintaining normal blood pressure in mice.

The present invention provides a protein composition that can be used to control blood pressure. The overexpression of this protein in transgenic mice lowered blood pressure and inhibited the induction of angiotensin II mediated hypertension. Transgenic mice, while treated with angiotensin II, had significantly higher relaxation of the mesenteric arteries in contrast to normally expressing wild-type mice. This natural protein or its derivatives can be used to control hypertension and pulmonary hypertension.

There are several distinct advantages to the protein drug over traditional hypertension therapies. First, the drug would be taken every 3-6 months as an injection rather than daily as a pill. Second, the drug is a naturally occurring human protein, which means there should not be any adverse side effects to treatment. Lastly, the protein drug has antioxidant properties that may have additional benefits for the heart. The protein represents a safer and healthier alternative with fewer side effects than current hypertension drugs.

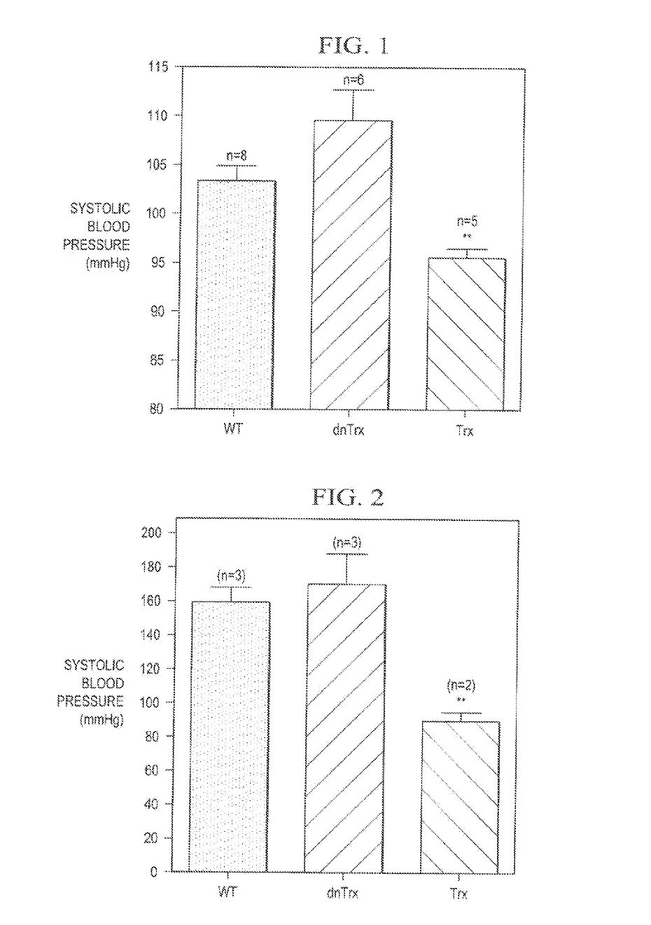

FIG. 1 shows Trx-Tg mice are phenotypes that show decreased blood pressure. The basal blood pressure measurements show that Trx-Tg mice have lower blood pressure than the wildtype or mice expressing mutant thioredoxin.

FIG. 2 shows thioredoxin lowers angiotensin II mediated hypertension in mice. Mice were infused with angiotensin II using osmotic minipumps for 14 days and the blood pressure was measured in time intervals. As shown in FIG. 2, the blood pressure measurement at the end of 10 days shows significant reduction in Trx-Tg mice compared to either wildtype or mutant mice. Additionally, the mutant mice show higher blood pressure compared to controls.

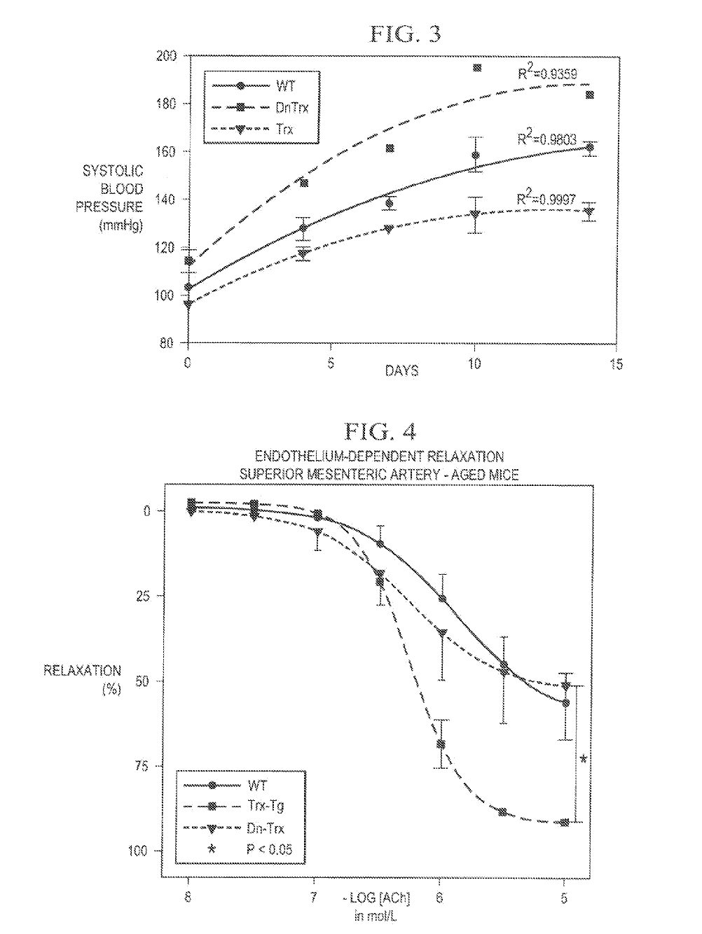

FIG. 3 shows the entire spectrum of 14 day blood pressure measurement that shows that in each time point the blood pressure of Trx-Tg mice is significantly lower.

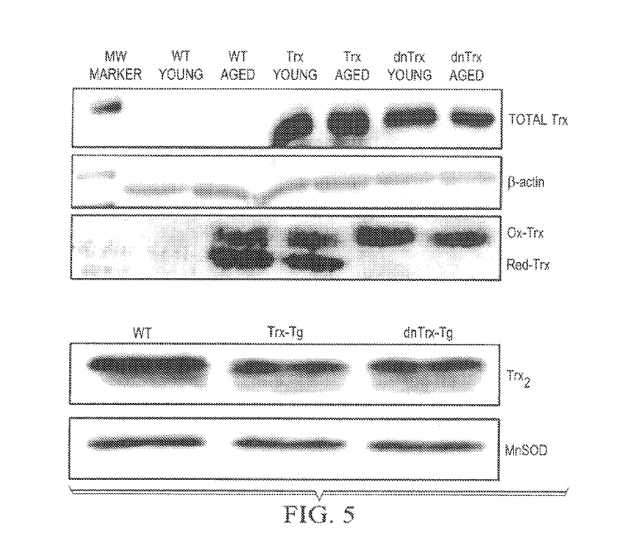

FIG. 4 shows the mesenteric artery isolated from the Trx-Tg mice show significantly higher relaxation response compared to wildtype or mutant mice. There is a significantly higher relaxation of mesenteric arteries isolated from Trx-Tg mice in response to Acetylcholine (Ach) compared to wildtype or mutant mice.

FIG. 5 is an image showing the redox state of vessels in WT, Trx-Tg and dnTrx-Tg in both young and old mice. The present data shows that aged Trx-Tg mice are normotensive, whereas aged dnTrx-Tg mice are more hypertensive compared to aged wildtype (wt) mice. Additionally, Trx-Tg mice maintain significantly lower blood pressure compared to aged wt or dnTrx-Tg mice. We show that increased levels of vascular Trx prevents age-related Trx oxidation observed in the vessels of Trx-deficient mice. Additionally, increased Trx levels maintained eNOS function in aged vessels in Trx-Tg mice, but not in dnTrx-Tg mice. The EC dysfunction observed in aged WT mice could be reversed by DTT, a chemical reductant. In contrast, DTT could not reverse EC dysfunction in aged vessels from dnTrx-Tg mice. Taken together, the cumulative oxidation of redox proteins in aging could be reversed by pharmacological intervention with Trx resulting in control of hypertension during aging.

The ionic composition of KRB solution was as follows (mM): 118.5 NaCl, 4.7 KCl, 2.5 CaCl2, 1.2 MgSO4, 1.2 KH2PO4, 25.0 NaHCO3, and 5.5 D-glucose. anti-eNOS (cat #610297) was obtained from BD Transduction Laboratories, USA. DAF-FM) diacetate (cat # D23844) was purchased from Life Technologies, Grand Island, N.Y. Attachment Factor (cat # S006100) was purchased from Life Technologies, Grand Island, N.Y. The MV bullet kit (cat # CC-3202) was obtained from Lonza, Walkersville, Md. All chemicals were purchased from Sigma-Aldrich.

Studies were conducted in young (2 to 5 months) and aged (20 to 28 months) wildtype, Trx-Tg and dnTrx-Tg mice. The background of these mice was C57BL/6J. All procedures were approved by the Institutional Animal Care and Use Committee at the Texas Tech University Health Sciences Center, and were consistent with the Guide for the Care and Use of Laboratory Animals published by the National Institute of Health.

Segments of SMA from aged WT mice were placed in 100 mL DMEM solution containing h-Trx-1 (1 g/L), h-Trx-1+TrxR (10 U), h-Trx-1+TrxR+NADPH (2 mM), or none. The four segments were placed in a CO2 incubator at 37° C. for 20 hours. The next day, segments were mounted on the wire-myograph and assessed for NO-mediated relaxing responses as described above.

The frozen carotid arteries were homogenized in 0.05 M potassium phosphate buffer (pH 7.0) containing 1 mM EDTA. Following homogenization in a Waring® blender, the homogenate was centrifuged in a microfuge at 14,000 rpm at 4.0° C. for 45 mins. The supernatant was transferred to another tube, and the Trx activity assay was performed immediately as described in our previous publications. Briefly, the reaction mixture was comprised of NADPH (200 μM) and porcine insulin (80 μM; Sigma) in 0.05 M potassium phosphate buffer (pH 7.0) containing EDTA (1 mM) in a total volume of 0.25 ml. The assay was standardized using E. coli Trx and rat thioredoxin reductase. The reaction was started by addition of rat thioredoxin reductase (0.1 μM). Trx activity was calculated as μmoles of NADPH oxidized per minute per mg protein at 25° C. Thioredoxin reductase activity was determined by the method as described by Holmgren et al. Peroxiredoxin activity was determined by the rate of decrease of NADPH using hydrogen peroxide as substrate. All assays were performed in Beckman DU800 spectrometer with Peltier temperature control and using quartz cuvettees.

Mesenteric arteries were excised from WT, Trx-Tg and dnTrx-Tg mice and cleaned of connective and adipose tissue. To generate sufficient protein to detect the oxidized or reduced endogenous murine Trx, mesenteric arteries from WT mice were pooled (6 from young and 6 from aged mice). Segments were homogenized in 0.5 mL of carboxymethylation buffer and the oxidized and reduced Trx levels were detected.

Mice were euthanized by an overdose of isofluorane and the mesentery was removed and placed in cold Krebs-Ringer Buffer (KRB). From each mouse, segments (2 mm) of the superior mesenteric artery (SMA) were carefully dissected and mounted in a wire-myograph (model 620M; Danish Myotechnology, Aarhus, Denmark) for the recording of isometric force development. SMA were incubated for 30 min in KRB with continuous aeration with 95% O2/5% CO2 and maintained at 37° C. SMAs were passively stretched according to a procedure first described by Halpern and Mulvany. In brief, vessels were distended stepwise, in 100 mm increments to their optimal lumen diameters for active tension development. The vessels were stretched to a passive wall tension of 90% of the internal circumference of that achieved when the vessels were exposed to a passive tension yielding a transmural pressure of 100 mmHg. At this passive wall tension segments were contracted with high K+ KRB (60 mM KCl in KRB solution; replacing equimolar NaCl with KCl), thus generating a stable contraction that reached a plateau after 10-15 min. This active wall tension was set to a 100% contraction level. After a 30 min washout period, cumulative concentration-response curves (CRC) were performed to the vasoconstrictor phenylephrine (PHE; 0.01-30 μM), which causes al-adrenergic-mediated contractions.

Endothelium-dependent relaxation responses to cumulative concentrations of acetylcholine (ACh; 0.01-10 μM) were determined in SMAs contracted with PHE (3-10 μM). To study NO-mediated relaxation responses, SMAs were treated with the non-selective cyclooxygenase blocker indomethacin (INDO; 10 μM) to inhibit vasodilator prostanoids. In addition, SMAs were treated with the selective endothelial Ca2+-activated K+ channel blockers (TRAM-34; 1 μM) and (UCL) 1684 (1 μM) to inhibit IK1 and SK3 channel activity, respectively, and subsequent inhibition of the endothelium-dependent hyperpolarization (EDH) relaxation. All three inhibitors were incubated for 30 min prior contraction with PHE. EDH relaxations were recorded with a combination of the non-selective NO synthase blocker Nω-Nitro-L-arginine methyl ester (L-NAME; 100 μM) and INDO (10 μM). Endothelium-independent relaxation responses to cumulative concentrations of the NO donor sodium nitroprusside (SNP; 0.1 nM-10 μM) were determined in SMA treated with L-NAME and INDO. In a subset of aged mice, SMAs were treated with dithiotreitol (DTT; 100 μM) for 30 min in the wire-myograph chamber before assessing ACh-mediated relaxing responses.

To study NO release in intact arteries, SMA from WT, Trx-Tg and dnTrx-Tg mice were freshly isolated, cleaned and cut open along its longitudinal axis. High level of care was applied to retain intact endothelium. Then the opened SMA were loaded with 4-amino-5-methylamino-2′,7′-difluorofluorescein (DAF-FM; 5 μM) for 5 min at 37° C. in oxygenated KRB and then treated with ACh (10 μM) for 5 seconds. After treatment, SMAs were fixed for 5 min in 3% paraformaldehyde and washed with PBS once and mounted using anti-fade mounting medium with endothelium facing the glass coverslip. Intracellular green fluorescence generated due to NO release was observed using Zeiss Axio Imager Z2 upright fluorescent microscope via 20×/0.8 NA objective.

Protein extracts were prepared from SMAs pooled from six mice. Segments were homogenized in cold PBS buffer in a glass douncer. The homogenate was centrifuged at 15,000 g for 15 min at 4° C. Supernatant (cystolic fraction) was kept on ice. The remaining pellet was resuspended in cold lysis buffer [50 mM TRIS.HCl (pH 7.4), 150 mM NaCl, 5 mmol/L EDTA, 1% Triton X-100, 50 mM NaF, 10 mM sodium pyrophosphate, 25 mM-glycerophosphate, 1 mM PMSF, 1 μg/mL leupeptin, 1 μg/mL aprotinin, 1 μg/mL pepstatin, and 1 mM Na3VO4]. The homogenate was centrifuged at 15,000 g for 20 min at 4° C. The supernatant (membrane fraction) was kept on ice. Protein concentration was determined with the BCA protein assay kit (Pierce Chemical, Rockford, Ill., USA).

Low-temperature PAGE (LT-PAGE) was performed for detection of eNOS dimers. Briefly, running buffer and 6% gels were cooled on ice prior to loading of 15 g of protein from either the membrane fraction or the cytosolic fraction. The buffer tank was placed on ice during electrophoresis to maintain the temperature of the gel below 15° C. The running buffer was changed every 30 min with ice-cold running buffer. After electrophoresis the gels were transferred onto nitrocellulose membrane, and membranes were blocked by treatment with 5% non-fat dry milk in Tris-buffered saline containing 0.05% Tween-20 (TBS-T), followed by incubation with primary antibody (anti-eNOS; BD Translab, cat #610296) overnight at 4° C. After incubation with secondary antibodies, signals were detected with chemiluminescence autoradiography.

FIG. 5 is an image showing the redox state of vessels in Wt, Trx-Tg and dnTrx-Tg in both young and old mice. The present data shows that aged Trx-Tg mice are normotensive, whereas aged dnTrx-Tg mice are more hypertensive compared to aged wildtype (WT) mice. Superior mesenteric arterial outward remodeling is enhanced with aging in Trx-Tg mice. The optimal diameters of segments of the main superior mesenteric artery (SMA) measured in the wire-myograph were comparable in young mice groups. The average diameter was 452±8 mm in WT, 425±11 mm in Trx-Tg, and 422±12 mm in dnTrx-Tg mice. Aging resulted in a statistically significant increase in lumen diameter in all the three mice groups compared to their younger counterparts. Interestingly, this outward remodeling was significantly larger in aged Trx-Tg (564±11 mm), compared to aged dnTrx-Tg mice 516±12 mm), but not to aged WT mice (543±14 mm).

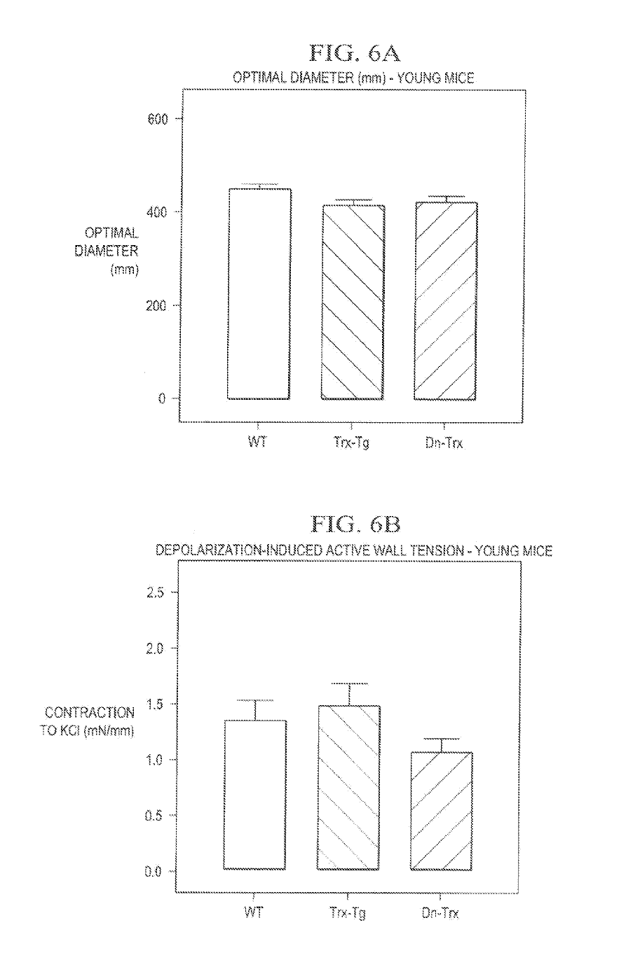

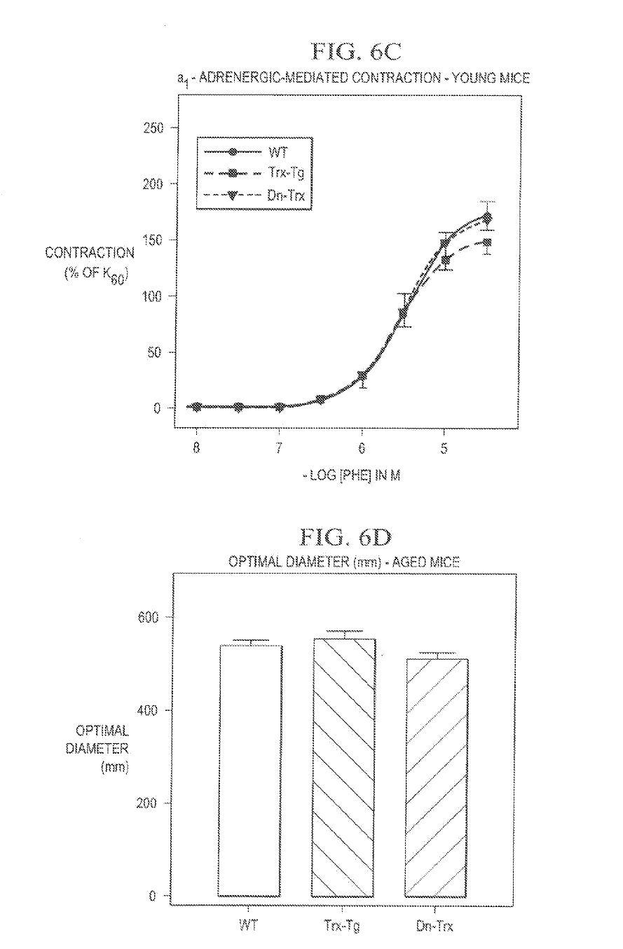

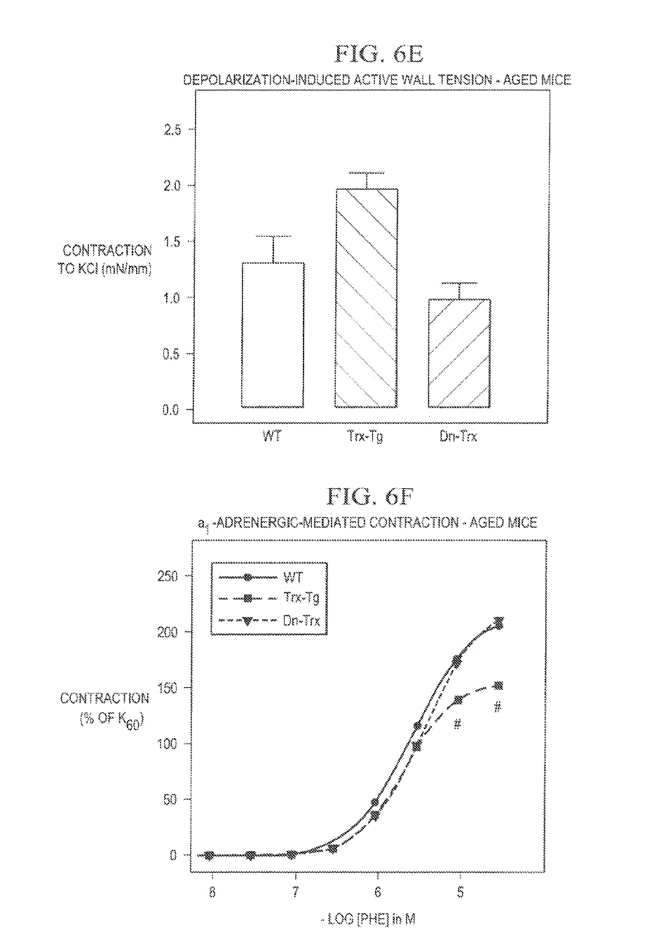

FIGS. 6A-6F show the SMA outward remodeling and depolarization- and agonist-induced contraction is maintained in Trx-Tg mice. FIGS. 6A-6D show optimal diameters, FIGS. 6B-6E show depolarization (60 mmol/L K+ KRB)-induced and FIGS. 6C-6F show phenylephrine-induced contraction for SMA derived from young (upper panel) and aged (lower panel) wild-type (white bars), Trx-Tg (blue bars) and dnTrx-Tg (red bars) mice. Values are means±SEM (n=8-10 mice). * P<0.05 compared with Trx-Tg. Addition of 60 mM K+ KRB resulted in a contraction due to opening of voltage-operated Ca2+ channels and subsequent transient contraction that reached a plateau after 10 to 15 min. Tensions did not differ between SMA derived from young mice. However, depolarization-induced contractions in SMA derived from aged Trx-Tg were statistically significantly larger compared to their WT and dnTrx-Tg counterparts. Phenylephrine (PHE) contracted SMA from young and aged mice in a concentration-dependent manner (FIGS. 6C and 6F). The sensitivity (pEC₅₀) to PHE did not differ significantly between arteries from young WT, Trx-Tg, and dnTrx-Tg mice (5.46±0.08, 5.56±0.12 and 5.48±0.07, respectively, FIG. 6E). The maximal tension (in mN/mm) generated by 30 milli molar PHE was also similar between arteries from young WT, Trx-Tg, and dnTrx-Tg mice (4.45±0.15, 4.05±0.19, and 4.04±0.12; respectively; FIG. 6C). Aging did not result in significant changes in pEC₅₀ values in the three mice groups (FIG. 6F), but maximal tension was statistically significantly increased in SMA from aged Trx-Tg mice (5.17±0.20) compared to their younger counterparts (FIG. 6F). In SMA from aged WT (4.87±0.34) and aged dnTrx-Tg (3.90±0.16) mice there was no statistically significant change in tension compared to their younger counterparts.

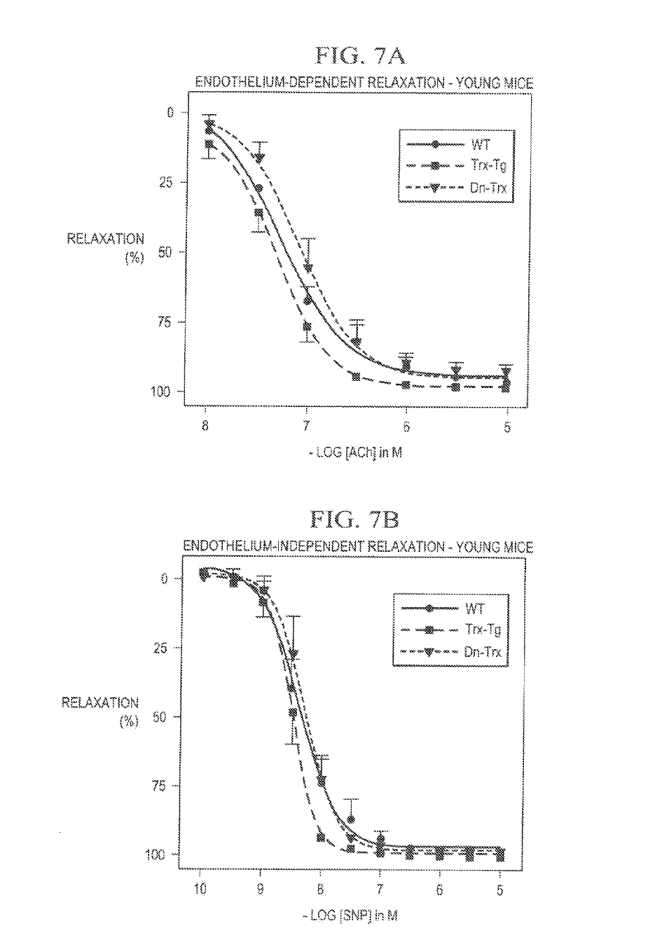

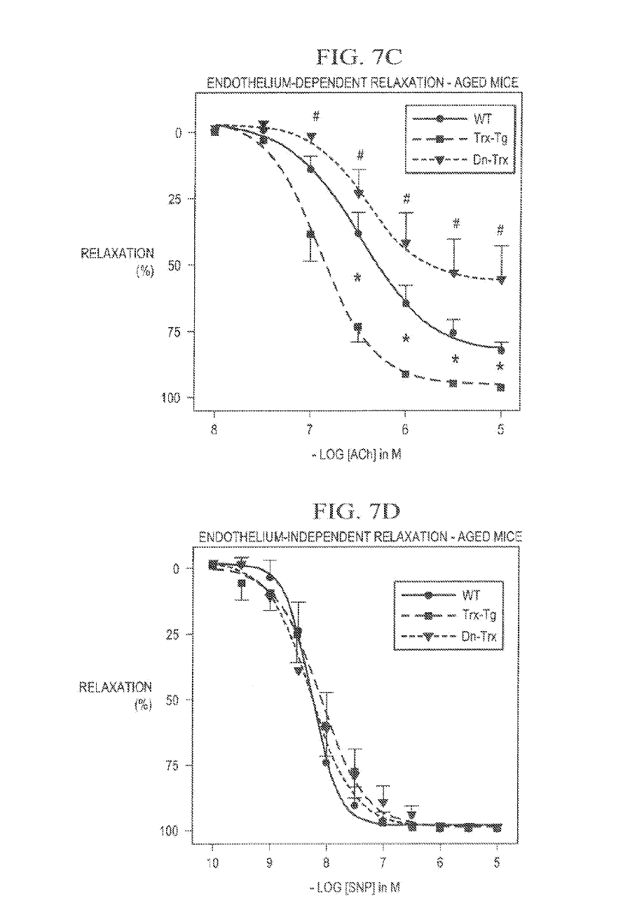

FIGS. 7A-7D show aging-induced endothelial dysfunction is suppressed in Trx-Tg mice compared to WT and dnTrx-Tg mice. FIGS. 7A-7D show aging-induced endothelial dysfunction is suppressed in Trx-Tg mice compared to WT and dnTrx-Tg mice. FIGS. 7A and 7C shows endothelium-dependent relaxations for SMA derived from young (FIG. 7A) and aged (FIG. 7B) wildtype (black circles), Trx-Tg (blue upward triangles) and dnTrx-Tg (red downward triangles) mice were contracted with a submaximal concentration of PHE before assessing relaxing responses to cumulative concentrations of ACh (0.01-10 mM). Endothelium-dependent relaxation was severely blunted with aging in DnTrx-Tg mice, but preserved in Trx-Tg mice. Endothelium-dependent acetylcholine (ACh)-mediated relaxations were reduced in SMA derived from young dnTrx-Tg mice compared to Trx-Tg mice (FIG. 7A). Sensitivity to ACh was statistically significantly decreased in SMA from dnTrx-Tg compared to Trx-Tg mice (6.88±0.13 versus 7.32±0.08), whereas sensitivity to ACh in SMA from young WT mice (7.02±0.11) was not different from either Trx-Tg or dnTrx-Tg mice. Maximal relaxation (Eₘₐₓ) in response to 10 μM ACh averaged 94±2%, 98±1%, and 92±3% in young WT, Trx-Tg, and dnTrx-Tg mice, respectively, which was comparable for all three groups. Aging resulted in a statistically significant right-ward shift in the concentration-response curves to ACh in all three mice groups (FIG. 7B). However, this shift was lower in Trx-Tg mice compared to WT and dnTrx-Tg mice, as evidenced by a statistically significantly greater pEC₅₀ value in aged Trx-Tg mice (6.74±0.06) compared to WT mice (6.35±0.09). Sensitivity for ACh in SMA from aged dnTrx-Tg mice could not be determined, since relaxations did not reach more than 50% in most SMA. Strikingly, Eₘₐₓ values to ACh were not different in SMA between young and aged Trx-Tg mice (98±1% versus 94±2%). This Eₘₐₓ for aged Trx-Tg mice was statistically significantly higher compared to aged dnTrx-Tg mice (47±11%), but not to aged WT mice (85±2%; FIG. 7B). FIG. 7C summarizes the relaxing responses from young and aged mice by depicted the calculated “area under the curve” values. The aging-induced changes were specific to the endothelium, since relaxing responses to the endothelium-independent NO donor sodium nitroprusside were comparable in SMA from both young (FIG. 7D) and aged (FIG. 7E) mice of all three groups.

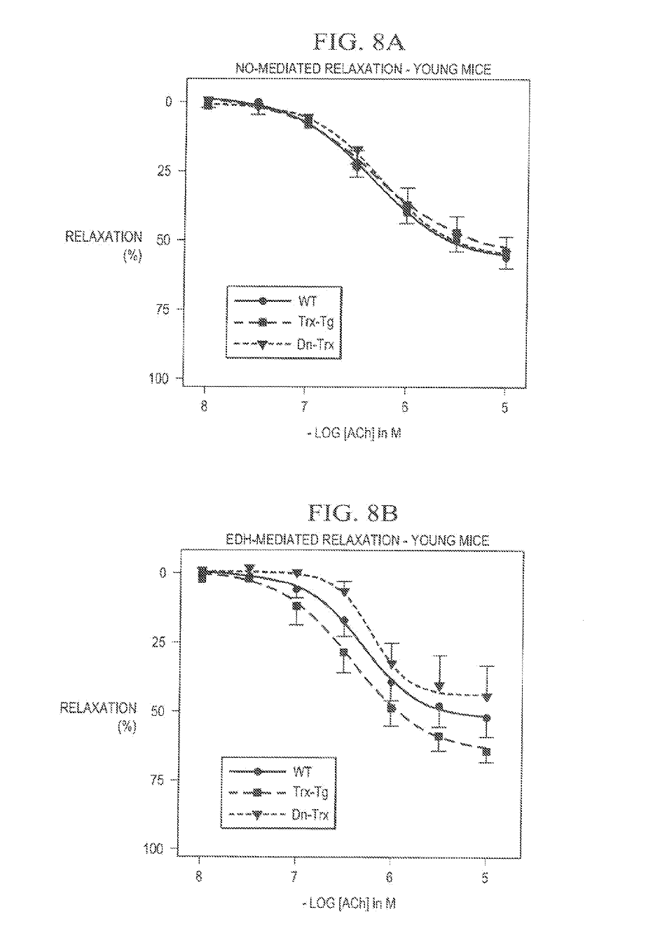

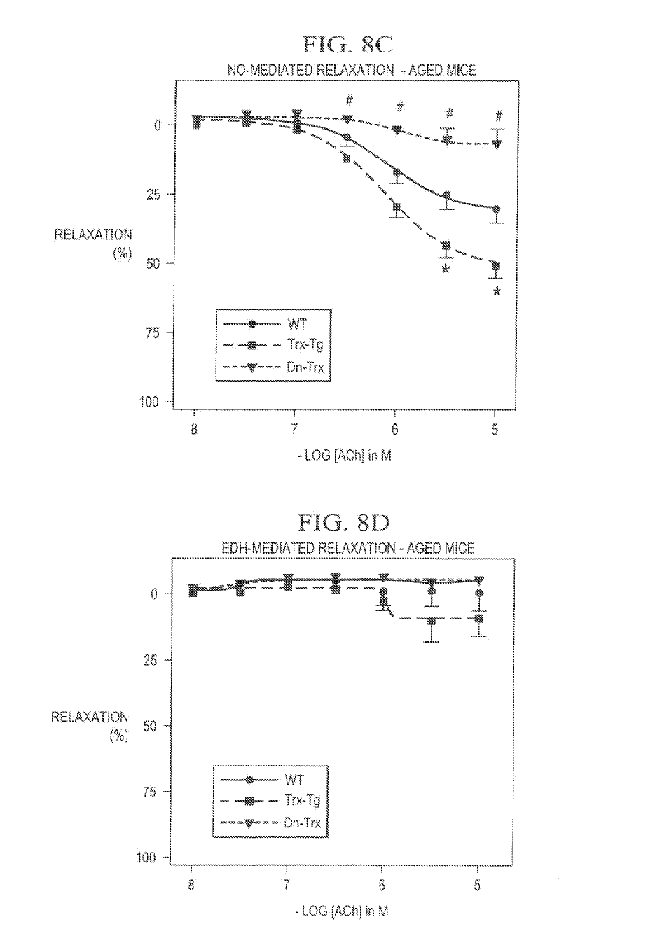

FIGS. 8A-8D show preserved NO-mediated relaxing responses and EDH-mediated responses in young and aged Trx-Tg mice, WT and dnTrx-Tg mice. NO-mediated endothelium-dependent relaxations in SMA derived from young (FIG. 8A) and aged (FIG. 8B) wildtype (black circles), Trx-Tg (blue upward triangles) and dnTrx-Tg (red downward triangles) mice were treated for 30 min with INDO (10 μM), and the combined presence of TRAM-34 (10 μM) and UCL 1684 (10 μM) to block vasodilator prostanoid release and EDH-mediated responses, respectively. SMA were contracted with a submaximal concentration of PHE, before assessing relaxing responses to cumulative concentrations of ACh (0.01-10 μM). FIGS. 8D and 8E show EDH-mediated responses in SMA derived from young (FIG. 8D) and aged (FIG. 8E) wildtype (black circles), Trx-Tg (blue upward triangles) and dnTrx-Tg (red downward triangles) mice were treated with L-NAME (100 mM) and INDO (10 mM) for 30 min, contracted with a submaximal concentration of PHE, and assessed with cumulative concentrations of ACh (0.01-10 μM). Values are means±SEM (n=6-8 mice). * P<0.05 versus Trx-Tg, # P<0.05 dnTrx-Tg versus WT and Trx-Tg, ** P<0.05 versus young mice. Since NO is the major endothelium-derived vasorelaxing factor in the superior mesenteric artery, we addressed the contribution of NO in ACh-mediated relaxations. To rule out vasorelaxing factors derived from cyclooxygenases, SMA were continuously treated with the non-selective cyclooxygenase inhibitor indomethacin (INDO; 10 μM). In addition, SMA were incubated with TRAM-34 (1 μM) and UCL 1684 (1 μM) to inhibit both IK1 and SK3 endothelial calcium-activated potassium channels, respectively to prevent endothelium-dependent hyperpolarization (EDH). Comparable NO-mediated relaxations were observed in SMAs from young mice (FIG. 8A). In SMA of Trx-Tg mice, NO-mediated relaxations were unaffected by advanced aging (FIG. 8B). However, WT and dnTrx-Tg mice showed markedly reduced NO-mediated relaxations with aging (FIGS. 8A and 8C). Next, we assessed EDH relaxations in the presence of L-NAME (100 μM) and INDO (10 μM). In young mice, comparable EDH responses were observed (FIG. 8B). Aging markedly blunted these EDH responses in SMAs from all three mice groups, but to a lesser extent in Trx-Tg mice. Based on the area under the curve values it is clear that NO is the major source of vasorelaxing factor in SMA.

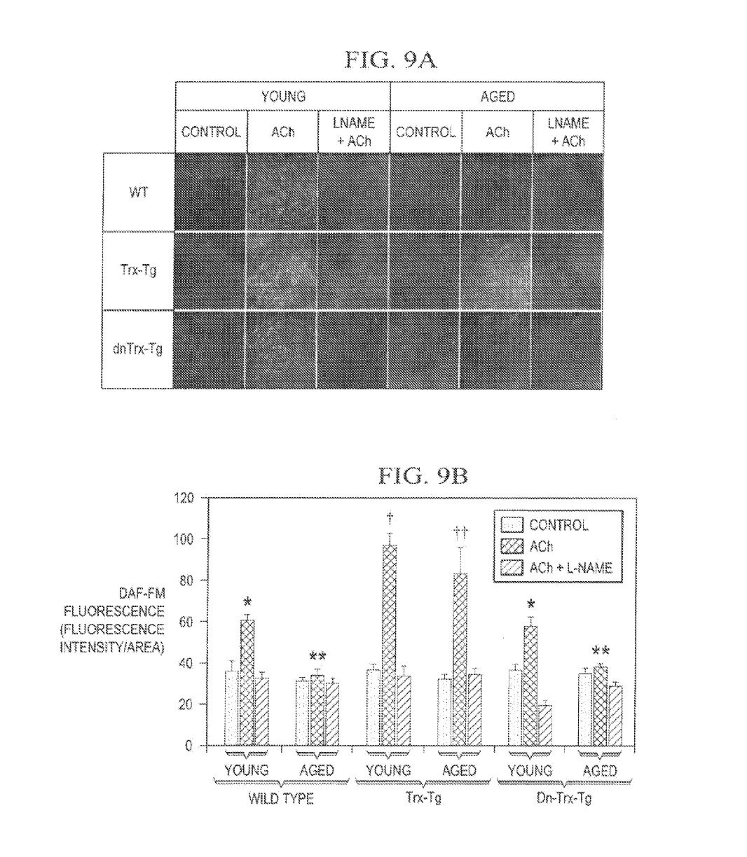

FIGS. 9A and 9B show Trx enhances ACh-mediated NO release in SMA from young and aged mice. FIG. 9A are fluorescence images of NO release by endothelium in longitudinally opened and en face arteries is visualized by loading arteries with 5 μM DAF-FM and treating with 10 μM ACh for 5 seconds. Images were captured using Zeiss Axio Imager Z2 upright fluorescent microscope via 20×/0.8 NA objective. In case of 1-NAME, arteries were first incubated with 100 μM 1-NAME for 30 min at 37° C., loaded with DAF-FM and treated with 10 μM ACh. All incubations and treatments were carried out in oxygenated KRB. FIG. 9B shows DAF-FM fluorescence upon NO release in the absence of ACh (black bars), presence of ACh (green bars) and ACh+1-NAME (red bars) was quantitated using AxioVision 4.9 software and bar graph represents mean±SD of mean fluorescence intensity of five arterial segments. * P<0.01 versus WT Young control; ** or † P<0.01 versus WT and Dn-Trx Young+ACh, †† P<0.01 versus WT and DnTrx-Tg Aged+ACh.

NO release in ex vivo mesenteric arteries is increased in Trx-Tg mice. We measured NO release in en face superior mesenteric arteries from young and aged WT, Trx-Tg and dnTrx-Tg mice using the NO probe DAF-FM followed by fluorescence microscopy. No statistically significant differences in basal NO release was measured in arteries derived from young mice. Incubation with ACh (10 μM) resulted in a rapid increase in green fluorescence, which was statistically significantly increased in Trx-Tg mice compared to WT and dnTrx-Tg mice. Pharmacological inhibition of NO synthesis using L-NAME (100 μM) prevented this increase in green fluorescence. Basal levels of NO release was unaltered with advanced aging in all mice groups and stimulation with ACh did not result in a significant increase in green fluorescence in arteries from aged WT and aged dnTrx-Tg mice. This in contrast to arteries from aged Trx-Tg mice, which clearly show a much higher fluorescent intensity after stimulation with ACh. Again, L-NAME blunted this effect.

Western blot of eNOS dimer and monomer in membrane fraction and cytosolic fraction of pooled mesenteric arteries from 6 young and 6 aged WT, Trx-Tg and dnTrx-Tg mice. Actin was used as internal standard. Densitometry analysis of eNOS dimer:monomer ratio from the blot. The ratio of dimer to monomer was normalized to the pixel intensity of actin. The ratio in young WT mice was set to 1. eNOS monomer was normalized to actin and set to 1 for WT Young mice. Densitometry analysis of eNOS monomer fragment of approximately 100 kD. eNOS monomer fragment was normalized to actin and set to 1 for WT Young mice.

NO release is triggered via eNOS dimer activation. We determined eNOS levels in both the membrane and cytosolic fraction from pooled (from n=6 mice) mesenteric arteries. The eNOS dimer was detected only in the membrane fraction under reducing conditions. Whereas in the cytosolic fraction predominantly the monomeric eNOS form was detected. The dimer to monomer ratios were calculated from the membrane fraction normalized to the actin band and compared to WT young mice. eNOS dimer:monomer ratio that decreases with aging in WT and dnTrx-Tg mice, but is maintained in Trx-Tg mice. In the cytosolic fraction, eNOS monomerization was increased with aging in WT and dnTrx-Tg mice, but unaltered in Trx-Tg mice, suggesting that Trx-1 maintains the eNOS dimerization for the increased NO production to protect endothelial dysfunction. Interestingly, a band of approximately 100 kDa predominantly appeared in cytosolic fractions of mesenteric arteries from aged dnTrx-Tg mice and that can be fairly detected in young mice.

Since aging is associated with increased oxidative stress we reasoned that Trx-1 could act as an antioxidant protein that would have beneficial effects on preserving endothelial function with advancing age. We used a method of carboxymethylation to dissociate between reduced and oxidized Trx-1. Fully reduced Trx-1 is carboxymethylated on two of its sulfhydryl groups located on two cysteine residues and migrates as a dicarboxylated band, and fully oxidized Trx-1 remains noncarboxylated and migrates slower on a native gel. Total human Trx-1 levels (molecular weight is 12 kD) in mesenteric artery lysates from pooled mice are shown. Faint bands corresponding to endogenous murine Trx-1 are shown for both young and aged WT bands. During aging the amount of total Trx-1 is slightly reduced compared to young mice. Mesenteric homogenates derived from young Trx-Tg mice, Trx-1 exists as both the reduced and oxidized form, with the reduced form being more predominant. In samples derived from aged Trx-Tg mice, the level of the reduced Trx-1 is lower compared to its young counterparts. In dnTrx-Tg mice, Trx-1 exists only in its oxidized form.

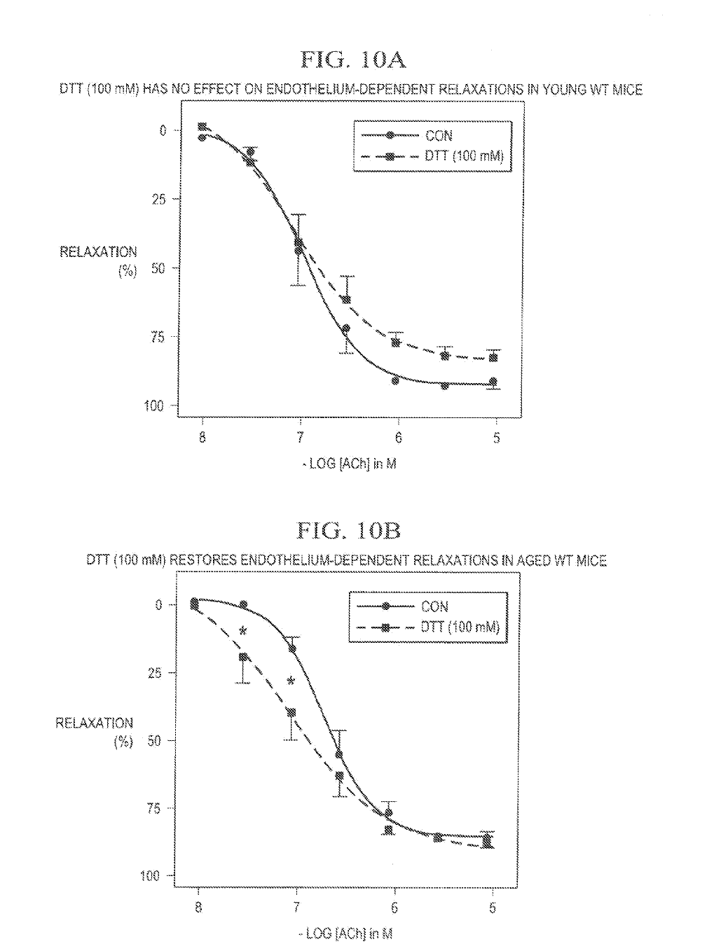

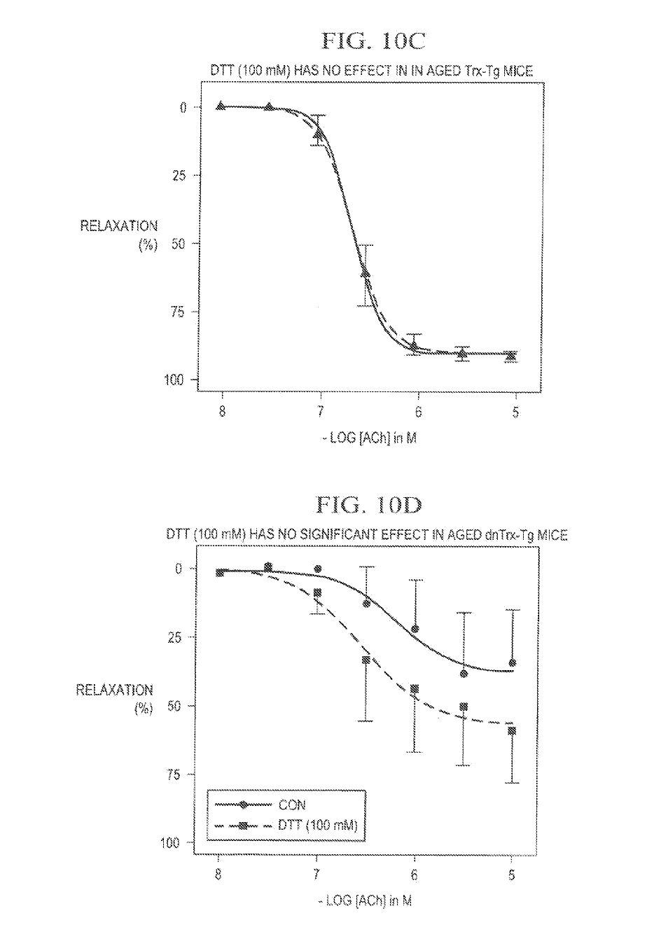

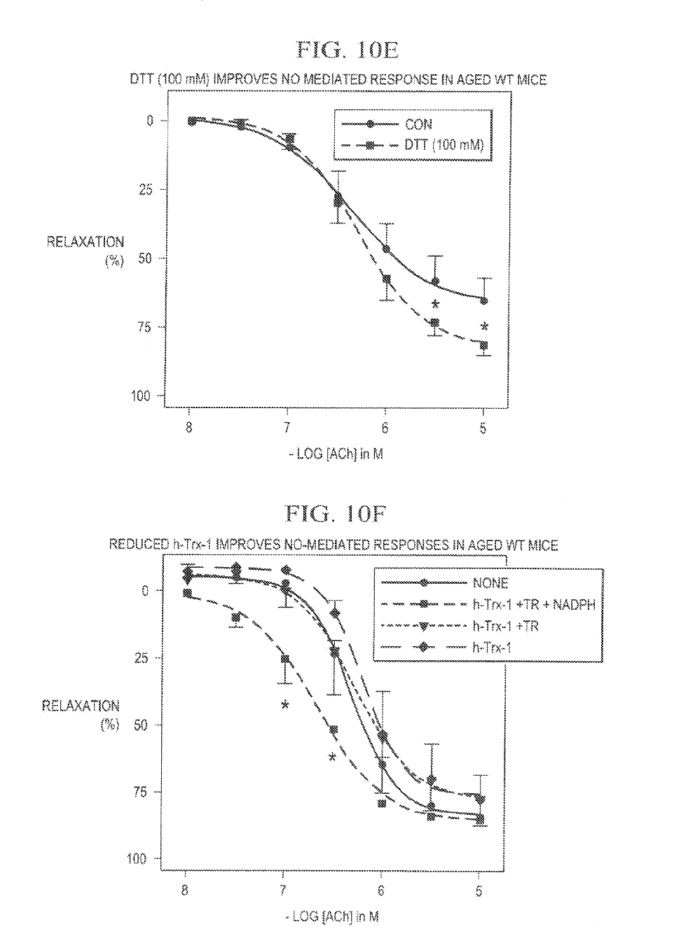

FIGS. 10A-10F show aging-induced endothelial dysfunction is restored by exogenous DTT and catalytically active h-Trx-1 in SMA from WT mice. Endothelium-dependent ACh-mediated relaxations in SMA derived from young WT (FIG. 10A), aged WT (FIG. 10B), Trx-Tg (FIG. 10C), and dnTrx-Tg (FIG. 10D) mice in the absence (CON) and presence of DTT (100 μM). (FIG. 10E) SMA from aged WT mice were incubated with INDO (10 μM), TRAM-34 (1 μM), and UCL1684 (1 μM) for 30 min in order to assess NO-mediated relaxing responses in the absence (CON) and presence of DTT (100 μM). (FIG. 10F) Cultured SMA were co-cultured with either a cocktail of catalytically active and recyclable h-Trx-1 (h-Trx-1+TR+NADPH), h-Trx-1+TR, h-Trx-1, or vehicle (None). SMA were contracted with a submaximal concentration of PHE before assessing relaxing responses to cumulative concentrations of ACh (0.01-10 μM). Values are means±SEM (n=3 mice). * P<0.05 versus CON, ** P<0.05 versus h-Trx-1+TR+NADPH. Since the active reduced Trx-1 is absent in dnTrx-Tg mice we questioned whether the protective effects in Trx-Tg mice were mediated by the sulfhydryl reducing property of reduced Trx-1. To address this we used the chemical sulfhydryl modifying agent DTT (100 μM) to mimic the effect of Trx-1. The aging-induced impairment in ACh-mediated relaxation in SMA from WT mice could be inhibited by incubating SMA for 30 min with DTT in the wire-myograph. Since the oxidized Trx-1 in dnTrx-Tg mice is mutated it cannot be reduced by DTT, but the endogenous murine Trx-1 can still be reduced by DTT. In SMA from young WT mice, DTT (100 μM) did not have an effect in modulating endothelium-dependent ACh-mediated relaxation (FIG. 10A). However, the same concentration of DTT restored ACh-mediated relaxations to comparable levels as in young mice (FIG. 10B). Sensitivity was statistically significantly increased in the presence of DTT (6.90±0.15) compared to in the absence of DTT (6.60±0.07). Strikingly, the sensitivity to ACh in the presence of DTT was not statistically significantly different from young mice (7.02±0.11; FIG. 10A). DTT had no effect in aged Trx-Tg mice (FIG. 10C), but tended to improve in dnTrx-Tg mice (FIG. 10D). DTT was able to improve NO-mediated responses in SMA from aged WT mice (FIG. 10E). Finally, we questioned whether culturing SMA overnight with h-Trx-1 would reverse the aging-induced endothelial dysfunction. Only the cocktail of h-Trx-1, TrxR and NADPH significantly improved NO-mediated relaxing responses (FIG. 10F). Interestingly, depletion of NADPH and h-Trx-1 alone resulted in comparable relaxing responses as SMA in the absence of h-Trx-1, indicating that the reduced h-Trx-1 is likely responsible for increased NO-mediated signaling.

The present invention provided compelling evidence to support that in transgenic mice, overexpressing the human sulfhydryl reducing Trx-1 protein (Trx-Tg), aging-induced endothelial dysfunction is protected compared to WT and mice expressing a dominant-negative mutant human Trx-1 (dnTrx-Tg). This observation is based on the following key findings include maintained NO-mediated relaxing responses, preserved functional NO release, protection of eNOS dimerization, and prevention of eNOS monomerization in superior mesenteric arteries derived from aged Trx-Tg mice. In addition we show that the chemical reducing agent DTT mimics the effect of Trx-1, suggesting that Trx-1 exerts these beneficial effects via its disulfide-reducing properties.

Contractile responses were markedly reduced in superior mesenteric arteries from aged dnTrx-Tg mice compared to both aged WT and Trx-Tg mice, as evidenced by a lower tension generated by a depolarizing high potassium chloride solution and in response to the al-adrenergic receptor agonist phenylephrine. Sensitivity to phenylephrine was not decreased with aging in all three mice groups. Vascular adrenergic nerve function has been shown not to decline in the rat mesenteric arterial bed, which is in agreement with our observations in mice. In small mesenteric arteries of the rat, the maximal tension in response to noradrenaline was comparable between young and aged animals, which are agreement with our findings in WT and dnTrx-Tg mice, but not Trx-Tg mice. However, this is the first study that reports aging-induced changes in murine superior mesenteric arterial structure and function. Internal diameters of superior mesenteric arteries were increased with aging, presumably due to enhanced blood flow demand in the gut with aging resulting in flow-induced arterial outward remodeling. Similarly, aging-induced increases in lumen diameter of murine second-order mesenteric arteries and rat carotid arteries have been reported. Despite this outward remodeling, maximal tensions to phenylephrine were comparable for young and aged WT and dnTrx-Tg mice, but increased in aged Trx-Tg mice compared to young Trx-Tg mice. One possible explanation for the unaltered contractility with aging in superior mesenteric arteries from WT and dnTrx-Tg mice might be the blunted aging-induced outward remodeling compared to Trx-Tg mice, which might imply a reduced wall mass and hence a decreased smooth muscle cell contractile capacity. NO plays a major role in arterial outward remodeling. In a surgical model of increased blood flow in the mesenteric artery increased eNOS expression and increased outward remodeling was observed, which was prevented by chronic eNOS inhibition by L-NAME and in eNOS knockout mice. It can therefore be anticipated that in aged WT and especially aged dnTrx-Tg mice, either functional NO release by shear stress is reduced and/or blood flow is decreased due to blunted NO-mediated vasodilatation. Both scenarios will result in blunted outward arterial remodeling. Cardiovascular diseases, such as hypertension, atherosclerosis, coronary artery disease, and also aging are characterized by endothelial dysfunction. In large arteries, where NO is the main vasorelaxing factor, the impairment in endothelium-dependent relaxation is manifested by a decrease in NO bioavailability, due to increased oxidative stress that uncouples the eNOS protein. Here, we confirm by multiple techniques that the aging-induced endothelial dysfunction is due to a reduction in NO bioavailability. We demonstrated a reduced ACh-mediated relaxing response in superior mesenteric arteries in aged mice compared to young mice, which was specific to the endothelium, since relaxing responses to the NO donor sodium nitroprusside, which activates soluble guanylate cyclase located in the smooth muscle cells, was unaltered with aging. When vasoactive prostanoids synthesis and the endothelium-dependent hyperpolarizing response were pharmacologically inhibited, the residual NO-mediated relaxations were decreased with aging in both WT and dnTrx-Tg mice, but were preserved in Trx-Tg mice. In addition, blunted endothelium-dependent hyperpolarizing (EDH) relaxing responses were observed with aging, which is in agreement with earlier observations in the rat mesenteric arterial bed. In superior mesenteric arteries from Trx-Tg mice, these EDH responses were less blunted compared to their WT and dnTrx-Tg counterparts.

We used the fluorescent NO probe DAF-FM to detect functional NO release in response to exogenously added ACh in en face arterial preparations. The specificity of this fluorescent technique was shown by the observation that the NOS inhibitor L-NAME prevented the NO release. Interestingly, a higher NO release was detected in arteries from young Trx-Tg mice compared to WT and dnTrx-Tg mice. Blunted NO release with aging was observed in arterial preparations from WT and dnTrx-Tg mice, which was preserved in Trx-Tg mice. These observations confirm our vascular reactivity results in that the ability of the endothelial cells in superior mesenteric arteries from Trx-Tg mice to generate NO in response to muscarinic receptor activation is protected with advanced age.

The above observations clearly show a reduced NO bioavailability with aging in WT and dnTrx-Tg mice, which was sustained in Trx-Tg mice. First we addressed the possibility that a reduced NO bioavailability might result from an increased eNOS monomerization. The active form of eNOS enzyme exists as two identical subunits and hence is located as a dimer in caseload. It has been shown that cysteines 94 and 99 of eNOS form a zinc tetra-coordinated (ZnS₄) cluster between each subunit. This ZnS₄ cluster is highly sensitive to oxidants such as ONOO⁺, NO, and H₂O₂ and the oxidation of this ZnS₄ cluster results in monomerization of eNOS and inhibition of catalytic activity. We have detected eNOS dimer and monomer forms using low-temperature SDS-PAGE using pooled protein lysate samples from the membrane fraction from 6 mice of each experimental group. The ratio between eNOS dimer to monomer was decreased in mesenteric arteries from aged WT and dnTrx-Tg mice, but was maintained in Trx-Tg mice. This reduction in eNOS dimers to monomers is in agreement with an earlier report studying endothelial dysfunction in mesenteric arteries from aged C57BL/6J mice. In the cytosolic fraction, increased eNOS monomerization was observed with aging in WT and dnTrx-Tg mice, but not in aged Trx-Tg mice. Concomitant with this increased eNOS monomerization an increased band of approximately 100 kD appeared in the mesenteric artery cytosolic fraction from aged dnTrx-Tg mice. This could be a cleaved eNOS product due to intracellular processing. Overall these data show that functional Trx-1 preserves eNOS dimerization, presumably via preventing the oxidation of the ZnS4 cluster.

A recent study by the group of Zweier revealed that oxidized glutathione altered eNOS activity via increased eNOS S-glutathionylation of two highly conserved cysteine residues resulting in eNOS uncoupling with superoxide anion release and impaired endothelium-dependent vasodilatation. This implies that redox-regulated mechanisms control eNOS function. Trx-1 contains two redox-active cysteine residues (Cys32 and Cys35) that can be reduced by receiving electrons from NADPH in the presence of Trx reductase. In this active state, reduced Trx-1 can reduce numerous oxidized proteins with disulfide bonds through thiol-disulfide exchange reactions. These two redox-active cysteine residues are mutated in dnTrx-Tg mice, which results that Trx-1 remains in its “oxidized” and catalytically inactive state in dnTrx-Tg mice. Here, we hypothesized that Trx-1 protects against aging-induced eNOS uncoupling via its disulfide-reducing properties in favor of reduced cysteine residues on proteins. To determine the redox state of the Trx-1 protein a chemical carboxymethylation method was used to detect both oxidized and reduced human Trx-1 in mesenteric artery lysates. As expected, only the oxidized human Trx-1 form was detectable in both young and aged dnTrx-Tg mice, whereas both oxidized and reduced human Trx-1 was present in Trx-Tg mice in about a 1:1 ratio. We speculate that the observed beneficial effects of human Trx-1 are mediated by the reduced and catalytically active form of Trx-1. This speculation is strengthened by our observation that DTT, which reduces disulfide bonds on cysteine residues, restores the aging-induced blunted endothelium-dependent ACh-induced relaxation in WT mice, whereas DTT had a marginal effect in aged Trx-Tg mice. This is the first study showing beneficial effects of Trx-1 in preserving aging-induced endothelial function at the level of the eNOS protein.

The present invention provides the administering of thioredoxin-1 to a subject in need of treatment for hypertension. Thioredoxin-1 is a different composition that thioredoxin-2 with different properties and characteristics. This is further illustrated in the fact that thioredoxin-1 functions differently and through a different pathway than thioredoxin-2. Thioredoxin-2 is mitochondrial and is not permeable into cells. It is transported to mitochondria by a transport mechanism. As a protein it cannot enter cells to have therapeutic effect. In contrast, thioredoxin 1 is cell permeable. From the data herein, the dnTrx mice have low levels of thioredoxin-1, but have regular levels of thioredoxin-2. But these mice show hypertension without any treatments such as angiotensin II infusion. Thus, removing thioredoxin-1 causes hypertension (dnTrxTg) and supplementing thioredoxin-1 decreases hypertension (Trx-Tg). Additionally, they specifically expressed Trx2 in the endothelial cells. We used global expression of Trx similar to injecting Trx that will go to every organ.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A pharmaceutical composition for treating a cardiovascular disorder comprising
(a) thioredoxin-1;
(b) a thioredoxin-1 derivative; or
(c) a combination of (a) and (b),
in a pharmaceutical carrier, in an amount effective to treat, or ameliorate one or more symptoms associated with, the cardiovascular disorder.

2. The pharmaceutical composition of claim 1, wherein the pharmaceutical carrier is one that is adapted for intramuscular, intraperitoneal, intravenous, or subcutaneous administration.

3. (canceled)

4. The pharmaceutical composition of claim 1, wherein the cardiovascular disorder is a hypertensive disorder.

5. (canceled)

6. A method for ameliorating one or more symptom of a cardiovascular disorder in a subject in need thereof comprising administering to the subject a therapeutically effective amount of a pharmaceutical composition according to claim 1, thereby ameliorating one or more symptom of said cardiovascular disorder.

7. The method of claim 6, wherein the pharmaceutical carrier is one that is adapted for intramuscular, intraperitoneal, intravenous, or subcutaneous administration.

8. The method of claim 6, wherein the pharmaceutical composition is administered every 0.5, 1, 2, 3, 4, 5, 6, or more months.

9. A method for treating a subject having a cardiovascular disorder comprising administering to the subject a therapeutically effective amount of
(a) a thioredoxin system upregulator or activator, and/or
(b) an agent that causes depletion of nitric oxide in the subject's body.

10. A method for (i) inhibiting aging-induced loss of endothelial cell function, (ii) preserving eNOS protein function or (iii) activating eNOS protein function in a subject, comprising administering to the subject a therapeutically effective amount of a pharmaceutical composition that comprises:
(a) thioredoxin-1
(b) a thioredoxin-1 derivative,
(c) a thioredoxin system upregulator, or
(d) a thioredoxin system activator,
in a pharmaceutical carrier, thereby inhibiting the aging-induced loss of endothelial cell function or preserving or activating eNOS protein function.

11. The method according to claim 10 for preserving eNOS protein function in said subject.

12. The method according to claim 10 for activating eNOS protein function in said subject.

13. The method of claim 6 wherein the cardiovascular disorder is a hypertensive disorder.

14. The method of claim 6, wherein the pharmaceutical carrier is one that is adapted for intramuscular, intraperitoneal, intravenous, or subcutaneous administration.

15. The method of claim 9 wherein the cardiovascular disorder is a hypertensive disorder.

16. The method of claim 9, wherein the pharmaceutical carrier is one that is adapted for intramuscular, intraperitoneal, intravenous, or subcutaneous administration.
